(19) United States
(12) Patent Application Publication
(10) Pub. No.: US 2002/0037302 A1
(43) Pub. Date: Mar. 28, 2002

(54) Composition in the form of a water-in-oil emulsion with a variable shear rate and methods of using the same

(75) Inventor: Isabelle Afriat, New York, NY (US)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'Oreal, Paris (FR)

(21) Appl. No.: 09/884,949

(22) Filed: Jun. 21, 2001

(30) Foreign Application Priority Data
Jun. 22, 2000 (FR) 0008011

(51) Int. Cl. A61K 7/00; C09K 3/00

(52) U.S. Cl. 424/401; 516/23

(57) ABSTRACT

The invention relates to a composition in the form of a water-in-oil emulsion containing at least 80% aqueous phase, containing a specific dimethicone copolyol as emulsifier, and comprising an oily phase/emulsifier weight ratio of greater than or equal to 5. The water preferably represents at least 66% of the total weight of the composition. The composition characteristically has a viscosity, measured using a Rheomat 180 viscometer at a shear rate of 200 s⁻¹ and at 25° C., ranging from 0.15 Pa·s (1.5 poises) to 20 Pa·s (200 poises). The composition according to the invention may have the appearance of a fluid or a cream and has specific rheological behavior: its shear rate changes over time, in particular when applied to the skin, where it “breaks” and promptly releases the aqueous phase, affording a great sensation of freshness. This composition may be used in particular in cosmetics and/or dermatology.

### CROSS REFERENCES TO RELATED APPLICATIONS

This application claims priority to French Patent Application No. 000801 1, filed on Jun. 22, 2000, and which is incorporated herein by reference in its entirety.

### BACKGROUND OF THE INVENTION

1. Field of the Invention

The present invention relates to compositions in the form of water-in-oil (W/O) emulsions which comprise a high water content and a specific silicone surfactant. The present invention further relates to compositions which have the appearance of a fluid or a cream and the use of such compositions in particular in cosmetics and/or dermatology.

2. Discussion of the Background

It is common practice in the fields of cosmetics and dermatology to use compositions which have the appearance of a fluid or a cream and consist of a water-in-oil (W/O) emulsion comprising an aqueous phase dispersed in an oily phase. In the fields under consideration, a cream or a fluid is a malleable and deformable product, as opposed to solid compositions.

W/O emulsions comprise an oily continuous phase and thus make it possible to form at the surface of the skin a lipid film which prevents transepidermal water loss and protects the skin against external attack. These emulsions are particularly suitable for protecting and nourishing the skin, and in particular for treating dry skin. However, W/O emulsions have the drawback, when applied to the skin, of giving it a fairly greasy feel, since the oily phase is the external phase. Thus, these emulsions are generally used for dry skin, since they are too greasy to be used on greasy skin. Furthermore, W/O emulsions afford no freshness and are generally too rich in oils to be used during summer or in hot countries.

To overcome these drawbacks, it has been envisaged to prepare emulsions with a high water content. However, the water content cannot be too high for reasons of stability. Alternatively, a high water content must be compensated for by adding several surfactants or gelling agents, which may harm the comfortable feel of the final composition and may even result in problems of skin irritation, especially in the case of individuals with sensitive skin.

European Patent Application EP-A-970 682 discloses compositions in the form of a stable water-in-oil emulsion comprising a large amount of water and containing as emulsifier a dimethicone copolyol comprising oxyethylenated groups and oxypropylenated groups. These compositions have a specific Rheological characteristic which makes them particularly advantageous for use in cosmetics. The reason for this is that, when applied to the skin, they “break,” that is to say that they suddenly become fluid due to the effect of shear, and they thus provide a great sensation of freshness on the skin. Such compositions are stable when stored at a temperature of 45° C. However, these compositions have the drawback of not having satisfactory stability in thermal storage cycles. A thermal storage cycle consists of subjecting the composition to several successive temperatures. Thus, the composition is maintained for a certain time (for example for 6 hours) at room temperature (about +20° C.) and then, over the same amount of time (i.e., 6 hours), the temperature is reduced to about −20° C. and the composition is left at this temperature of −20° C. for the same amount of time again (i.e., 6 hours), the temperature is then returned to room temperature (+20° C.) for the same amount of time (6 hours), and this process is repeated several times (generally 5 times). This passage through different temperatures makes it possible to test the full stability of a composition. Since it is advantageous for cosmetic compositions to have excellent stability, irrespective of the conditions under which they are maintained, there remains a need for water-in-oil emulsions which have a high water content and are stable even when stored under conditions in which the temperature fluctuates

### SUMMARY OF THE INVENTION

Accordingly, it is one object of the present invention to provide novel water-in-oil emulsions.

It is another object of the present invention to provide novel water-in-oil emulsions which have a high water content.

It is another object of the present invention to provide novel water-in-oil emulsions which have a high water content and which are stable even when stored under conditions of fluctuating temperature.

It is another object of the present invention to provide novel compositions which contain such a water-in-oil emulsion in a physiologically acceptable medium.

It is another object of the present invention to provide novel compositions, which contain such a water-in-oil emulsion in a physiologically acceptable medium, and which “break” upon application to the skin.

It is another object of the present invention to provide novel compositions, which contain such a water-in-oil emulsion in a physiologically acceptable medium, and which are useful for application to greasy skin.

It is another object of the present invention to provide novel methods for treating, protecting, caring for, removing make-up from and/or cleansing the skin, the lips and/or the hair, and/or for making up the skin and/or the lips by applying such a water-in-oil composition to the skin, the lips, and/or the hair.

It is another object of the present invention to provide novel methods for treating, protecting, caring for, removing make-up from, making up, and/or cleansing greasy skin, by applying such a water-in-oil composition to greasy skin.

These and other objects, which will become apparent during the following detailed description, have been achieved by the inventor's discovery that dimethicone copolyols free of oxypropylenated groups, i.e. containing only oxyethylenated groups, make it possible to obtain water-in-oil emulsions which contain a large amount of water and have the same rheological properties as those disclosed in European Patent Application EP-A-970 682, while at the same time having excellent thermal cycle stability.

### DETAILED DESCRIPTION OF THE PREFERRED EMBODIMENTS

Thus, in a first embodiment, the present invention provides water-in-oil emulsions, comprising an aqueous phase dispersed in an oily phase with the aid of a silicone emulsifier, wherein: (1) the aqueous phase represents at least 80% by weight relative to the total weight of the water-in-oil emulsion; (2) the oily phase/emulsifier weight ratio is greater than or equal to 5; and (3) the silicone emulsifier is a dimethicone copolyol comprising only oxyethylenated groups.

In a second embodiment, the present invention provides compositions comprising, in a physiologically acceptable medium, an aqueous phase dispersed in an oily phase with the aid of a silicone emulsifier, wherein: (1) the aqueous phase represents at least 80% by weight relative to the total weight of the composition; (2) the oily phase/emulsifier weight ratio is greater than or equal to 5; and (3) the silicone emulsifier is a dimethicone copolyol comprising only oxyethylenated groups.

In the compositions of the present invention, the expression “physiologically acceptable medium” means a non-toxic medium which may be applied to the skin (including the inside of the eyelids) or the lips of humans.

The composition obtained according to the present invention generally has a viscosity of greater than 0.15 Pa·s (1.5 poises) and preferably ranging from 0.2 Pa·s (2 poises) to 20 Pa·s (200 poises). This viscosity is measured using a Rheomat 180 machine, that is to say with an RM180 Rheomat machine from the company Mettler, generally at a shear rate of 200 s⁻¹ and at a temperature of 20 to 25° C.

The composition according to the present invention comprises at least 80% by weight and preferably at least 81% by weight of aqueous phase relative to the total weight of the composition. The aqueous phase may constitute up to 91% of the total weight of the composition.

Water constitutes at least 66% and preferably at least 70% of the total weight of the composition.

The aqueous phase of the emulsion may contain one or more lower alcohols such as ethanol in an amount which may be up to 15% and better still up to 10% of the total weight of the composition. Moreover, this aqueous phase may advantageously contain one or more polyols such as glycerol and glycols such as propylene glycol, in an amount ranging, for example, up to 20% and better still up to 10% of the total weight of the composition.

The composition of the present invention contains as an emulsifier a dimethicone copolyol comprising, as oxyalkylenated groups, only oxyethylenated groups, such as (polyoxyethylene)polydimethylsiloxanes or dimethyl(polyoxyethylene)polysiloxanes, or dimethylsiloxane methyl(polyoxyethylene)siloxane coploymers. Such a dimethicone copolyol is free of oxypropylenated groups. Dimethicone copolyols are non-crosslinked polydimethylsiloxane polymers. Suitable dimethicone copolyols comprise from 1 to 100, preferably from 20 to 30, dimethylsiloxy, —OSi(CH₃)₂—, units and contain from 1 to 10, preferably 2 to 6, ethyleneoxy, —OCH₂CH₂— units. A preferred class of dimethicone copolyols has the general formula:

(CH₃)₃Si—[OSi(CH₃)₂—]ₙ—[OSiR(CH₃)—]ₘ—OSi(CH₃)₃,

where n is a number from 1 to 100, preferably from 20 to 30, and m is a number from 1 to 10, preferably from 2 to 5, and R is a group of the formula:

—O—(CH₂CH₂O)ₚ—H

where p is a is a number from 1 to 10, preferably from 2 to 5. The product sold under the trade name “KF-6015” by the company Shin-Etsu may be used in particular in the composition according to the invention as a dimethicone copolyol.

The emulsifier is preferably present in an amount of active material ranging from 0.5% to 5% and better still from 0.6% to 4% by weight relative to the total weight of the composition.

Preferably, the composition of the present invention is free of any other emulsifiers than the dimethicone copolyol emulsifiers specified above. Even when the composition is free of any other emulsifier, it has excellent stability over time.

The weight ratio of the oily phase to the emulsifier (as active material) is greater than or equal to 5 and preferably greater than or equal to 8. The term “oily phase” means herein the amount of all the oily constituents, except for the amount as active material of silicone surfactant.

The oily phase of the composition according to the present invention may contain oils and fatty substances of any kind that are well known to those skilled in the art, for example oils of plant origin (jojoba oil, avocado oil, sesame oil, sunflower oil, corn oil, soybean oil, safflower oil, or grape pip oil), mineral oils (petroleum jelly, optionally hydrogenated isoparaffins), synthetic oils (isopropyl myristate, cetearyl octanoate, polyisobutylene, ethylhexyl palmitate or alkyl benzoates), volatile or non-volatile silicone oils such as polydimethylsiloxanes (PDMSs) and cyclodimethylsiloxanes or cyclomethicones, and fluoro oils or fluorosilicone oils, as well as mixtures of these oils.

Preferably, the oily phase of the composition of the present invention comprises at least one volatile silicone oil. The volatile silicone oil(s) may be present in an amount of at least 5% by weight and preferably ranging from 5% to 25% by weight relative to the total weight of the composition. Volatile silicone oils which may be mentioned, for example, include cyclic silicones such as pentacyclomethicone, tetracyclomethicone or hexacyclomethicone. According to one particular embodiment of the present invention, the oily phase consists solely of one or more volatile silicone oils.

The oily phase may also contain other fatty constituents such as fatty alcohols, for instance stearyl alcohol, cetyl alcohol and a mixture thereof (cetearyl alcohol), and fatty acids.

The composition of the present invention is preferably free of waxes.

The oily phase is present in the composition according to the present invention in an amount ranging from 8.5% to 21.5% and preferably from 10% to 18% by weight relative to the total weight of the composition.

The composition according to the present invention may contain a large amount of electrolyte without its stability being harmed.

Electrolytes which may be mentioned, for example, include mono-, di- and trivalent metal salts, and more particularly alkaline-earth metal salts such as barium, calcium, and strontium salts; alkali metal salts such as sodium and potassium salts, magnesium, beryllium, yttrium, lanthanum, cerium, praseodymium, neodymium, promethium, samarium, europium, gadolinium, terbium, dysprosium, holmium, erbium, thulium, ytterbium, lutetium, lithium, tin, zinc, manganese, cobalt, nickel, iron, copper, rubidium, aluminium, silicon, and selenium salts; and mixtures thereof.

The ions constituting these salts may be chosen, for example, from carbonates, bicarbonates, sulfates, glycerophosphates, borates, chlorides, bromides, nitrates, acetates, hydroxides, and persulphates, as well as the salts of α-hydroxy acids (citrates, tartrates, lactates, or malates) or of fruit acids, or alternatively the salts of amino acids (aspartate, arginate, or glycocholate) or fumarates.

The electrolyte is preferably a mixture of salts in particular comprising calcium, magnesium and sodium salts, and in particular a mixture comprising at least magnesium chloride, potassium chloride, sodium chloride, calcium chloride and magnesium bromide, the said mixture corresponding to Dead Sea salts.

The content of electrolyte, when it is present, generally ranges from 0.5% to 20% and preferably from 1% to 10% by weight relative to the total weight of the composition.

The composition according to the present invention contains a physiologically acceptable medium and may in particular constitute a cosmetic or dermatological composition. It finds its application in a large number of treatments, in particular cosmetic treatments of the skin, including the scalp, the hair, the nails and/or mucuous membranes, in particular for caring for and/or making up and/or antisun-protecting the skin and/or mucous membranes, and also for preparing a composition for treating the skin, more particularly greasy skin (provision of a sensation of freshness).

Thus, in another embodiment, the present invention provides a method of using the composition as defined above, for treating, protecting, caring for, removing make-up from and/or cleansing the skin, the lips and/or the hair, and/or for making up the skin and/or the lips.

In another embodiment, the present invention provides a method for treating the skin, including the scalp, the hair and/or the lips, characterized in that a composition as defined above is applied to the skin, the hair and/or the lips.

In another embodiment, the present invention a method of using the composition as defined above for treating greasy skin.

In a known manner, the composition of the present invention may also contain adjuvants that are common in cosmetics and/or dermatology, such as active agents, preserving agents, antioxidants, complexing agents, solvents, fragrances, fillers, bactericides, odor absorbers, dyestuffs, and lipid vesicles. The amounts of these various adjuvants are those conventionally used in the field under consideration, and, for example, from 0.01% to 20% of the total weight of the composition. Depending on their nature, these adjuvants may be introduced into the fatty phase, into the aqueous phase and/or into lipid vesicles.

In addition to the electrolytes mentioned above, active agents which may be mentioned in particular include moisturizers and, for example, protein hydrolysates and polyols such as glycerol, glycols, for instance polyethylene glycols, and sugar derivatives; natural extracts; procyannidol oligomers; vitamins; urea; depigmenting agents such as kojic acid and caffeic acid; β-hydroxy acids such as salicylic acid and its derivatives; α-hydroxy acids such as lactic acid and glycolic acid; retinoids such as carotenoids; screening agents; and mixtures thereof.

The active agent(s) may be present, for example, in a concentration ranging from 0.01% to 20%, preferably from 0.1% to 5% and better still from 0.5% to 3% relative to the total weight of the composition.

As noted above, the present compositions may be used for treating, protecting, caring for, removing make-up from, and/or cleansing the skin, the lips, the scalp, and/or the hair, and/or of making up the skin and/or the lips by applying the composition to the skin, lips, scalp, and/or hair. When using the present composition in such a manner, the composition is suitably applied to the skin, lips, scalp, and/or hair in an amount of 0.1 to 5 mg/cm², preferably 0.5 to 4 mg/cm² , more preferably 1 to 3 mg/cm² (these numbers being approximate). The amount is generally 2 mg/cm².

The present water-in-oil emulsions may be prepared by any conventional technique and with any apparatus conventionally used to prepare water-in-oil emulsions. Suitably, the present water-in-oil emulsions may be prepared by separately forming: (1) the oily phase, including the dimethicone polyol emulsifier; and (2) the aqueous phase, and then mixing the oily and aqueous phases.

Other features of the invention will become apparent in the course of the following descriptions of exemplary embodiments which are given for illustration of the invention and are not intended to be limiting thereof.

### EXAMPLES

In the following examples, and throughout this specification, all amounts are percentages by weight, and all temperatures are in degrees Celsius, unless expressly stated to be otherwise.

### Example

### Care Fluid

| A. | Oily phase | |
|---|---|---|
| | Dimethicone copolyol (KF-6015) | 1.75% |
| | Pentacyclomethicone | 17.75% |
| B. | Aqueous phase | |
| | Sodium chloride | 2.5% |
| | Glycerol | 7% |
| | Water | 71% |

Procedure: the two phases are prepared separately, and the aqueous phase is introduced into the oily phase.

A white fluid with a viscosity, measured using a Rheomat 180 machine, of 2.7 poises (0.27 Pa·s) at time zero is obtained. This viscosity stabilizes after 10 minutes at 2.54 poises (0.254 Pa·s).

This composition is stable when subjected to cycles over 5 days (6 hours at +20° C., 6 hours to go down to −20° C., 6 hours at −20° C., 6 hours to return to +20° C., and repetition of the cycle 5 times). This passage through different temperatures makes it possible to test the full stability of a composition.

### Comparative Examples 1 and 2

In the composition of the example given above, the KF-6015 is replaced with a silicone surfactant comprising oxyethylenated groups and oxypropylenated groups, and these compositions are subjected to cycles over 5 days as for the composition according to the invention.

| | Example | | |
|---|---|---|---|
| | according to | Comparative | Comparative |
| | the invention | Example 1 | Example 2 |
| Silicone | KF-6015 | DC2-5185 C (Dow | Silwet FZ-2108 |
| surfactant | | Corning) | (Witco) |
| Viscosity | 2.54 poises | 18.8 poises | 4.5 poises |
| (at a time | (0.254 Pa · s) | (0.188 Pa · s) | (0.45 Pa · s) |
| of 10 min) | | | |
| Stability | good | destabilization after | destabilization after |
| in cycles | | 2 days (appearance | 2 days (phase |
| over 5 days | | of drops of oil) | separation) |
| Stability | good | good | good |
| after one | | | |
| week at | | | |
| 45° C. | | | |

The DC2-5185 C and Silwet FZ-2108 used in Comparative Examples 1 and 2 are dimethicone copolyols comprising both oxyethylenated groups and oxypropylenated groups. DC2-5185 C comprises 18 oxyethylenated groups and 18 oxypropylenated groups, and Silwet FZ-2108 comprises a ratio of the oxyethylenated groups to the oxypropylenated groups of 33:67.

This table shows that only the silicone surfactant comprising only oxyethylenated groups used according to the invention gives good cycle stability.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A water-in-oil emulsion, comprising an aqueous phase dispersed in an oily phase with the aid of a silicone emulsifier, wherein:
(1) said aqueous phase is present in an amount of at least 80% by weight relative to the total weight of said oil-in-water emulsion;
(2) said oily phase and said emulsifier are present in a weight ratio of said oily phase to said emulsifier greater than or equal to 5; and
(3) said emulsifier is a dimethicone copolyol comprising only oxyethylene groups as oxyalkylene groups.

2. A composition, comprising, in a physiologically acceptable medium, an aqueous phase dispersed in an oily phase with the aid of a silicone emulsifier, wherein:
(1) said aqueous phase is present in an amount of at least 80% by weight relative to the total weight of said composition;
(2) said oily phase and said emulsifier are present in a weight ratio of said oily phase to said emulsifier greater than or equal to 5; and
(3) said emulsifier is a dimethicone copolyol comprising only oxyethylene groups as oxyalkylene groups.

3. The composition according to claim 2, which has a viscosity, measured using a Rheomat 180 viscometer at a shear rate of 200 s⁻¹ and at 25° C., ranging from 0.15 Pa·s to 20 Pa·s.

4. The composition according to claim 2, which comprises at least 70% water relative to the total weight of the composition.

5. The composition according to claim 2, wherein said emulsifier is present in an amount ranging from 0.5% to 6% by weight relative to the total weight of the composition.

6. The composition according to claim 2, wherein said oily phase is present in an amount ranging from 10% to 18% by weight relative to the total weight of the composition.

7. The composition according to claim 2, said oily phase and said emulsifier are present in a weight ratio of said oily phase to said emulsifier greater than or equal to 8.

8. The composition according to claim 2, wherein said oily phase comprises one or more volatile silicone oils.

9. A method for treating, protecting, caring for, removing make-up from and/or cleansing the skin, the lips and/or the hair, and/or for making up the skin and/or the lips, said method comprising:
applying a composition, comprising, in a physiologically acceptable medium, an aqueous phase dispersed in an oily phase with the aid of a silicone emulsifier, wherein:
(1) said aqueous phase is present in an amount of at least 80% by weight relative to the total weight of said composition;
(2) said oily phase and said emulsifier are present in a weight ratio of said oily phase to said emulsifier greater than or equal to 5; and
(3) said emulsifier is a dimethicone copolyol comprising only oxyethylene groups as oxyalkylene groups.
to the skin, the lips and/or the hair of a subject in need thereof.

10. The method of claim 9, wherein said composition has a viscosity, measured using a Rheomat 180 viscometer at a shear rate of 200 s⁻¹ and at 25° C., ranging from 0.15 Pa·s to 20 Pa·s.

11. The method of claim 9, wherein said composition comprises at least 70% water relative to the total weight of the composition.

12. The method of claim 9, wherein said emulsifier is present in an amount ranging from 0.5% to 6% by weight relative to the total weight of the composition.

13. The method of claim 9, wherein said composition comprises said oily phase in an amount ranging from 10% to 18% by weight relative to the total weight of the composition.

14. The method of claim 9, wherein in said composition said oily phase and said emulsifier are present in a weight ratio of said oily phase to said emulsifier greater than or equal to 8.

15. The method of claim 9, wherein in said composition said the oily phase comprises one or more volatile silicone oils.

16. A method for treating, protecting, caring for, removing make-up from, making up, and/or cleansing greasy skin, said method comprising:
applying a composition, comprising, in a physiologically acceptable medium, an aqueous phase dispersed in an oily phase with the aid of a silicone emulsifier, wherein:
(1) said aqueous phase is present in an amount of at least 80% by weight relative to the total weight of said composition;
(2) said oily phase and said emulsifier are present in a weight ratio of said oily phase to said emulsifier greater than or equal to 5; and
(3) said emulsifier is a dimethicone copolyol comprising only oxyethylene groups as oxyalkylene groups.
to greasy skin of a subject in need thereof.

17. The method of claim 16, wherein said composition has a viscosity, measured using a Rheomat 180 viscometer at a shear rate of 200 s⁻¹ and at 25° C., ranging from 0.15 Pa·s to 20 Pa·s.

18. The method of claim 16, wherein said composition comprises at least 70% water relative to the total weight of the composition.

19. The method of claim 16, wherein said emulsifier is present in an amount ranging from 0.5% to 6% by weight relative to the total weight of the composition.

20. The method of claim 16, wherein said composition comprises said oily phase in an amount ranging from 10% to 18% by weight relative to the total weight of the composition.

21. The method of claim 16, wherein in said composition said oily phase and said emulsifier are present in a weight ratio of said oily phase to said emulsifier greater than or equal to 8.

22. The method of claim 16, wherein in said composition said the oily phase comprises one or more volatile silicone oils.
